(19) United States
(12) Patent Application Publication
(10) Pub. No.: US 2006/0062815 A1
(43) Pub. Date: Mar. 23, 2006

(54) Therapeutic glove

(76) Inventor: Tony K. Djie, Encino, CA (US)

Correspondence Address: David S. Park;MacPHERSON KWOK CHEN & HEID LLP, Suite226, 1762 Technology Drive, San Jose, CA, 95110, US

(21) Appl. No.: 10/943,292

(22) Filed: Sep. 17, 2004

(51) Int. Cl. A61K 9/70; A61K 36/42

(52) U.S. Cl. 424/402; 424/443; 424/758

(57) ABSTRACT

A protective and therapeutic glove includes a coating of cucumber extract and/or glycerin on an interior surface. The coating, in contact with perspiration from a hand wearing the glove, soothes the hand. Some methods of placing the coating onto the inside surface of the glove include spraying with or dipping in a solution that includes cucumber extract and/or glycerin.

### BACKGROUND

1. Field of Invention

This invention relates to gloves, and more particularly to a glove with therapeutic properties for the wearer.

2. Discussion of the Related Art

Disposable gloves, for example, disposable examination gloves, are widely used as a protective measure to insulate hands from the objects handled by the wearer of gloves. To allow ease in handling objects, disposable gloves are made of thin and elastic material to minimize the space between the skin and the glove. Due to poor air circulation resulting from tight insulation, hand sweating is a common problem among glove wearers. Itchiness is a frequent result of wearing disposable examination gloves for extended periods of time.

Powders are commonly used on the inner surface of gloves to alleviate sweating and to make donning, wearing, and/or removing of gloves easier. However, continuous sweating can easily overwhelm the thin layer of powder that is commonly attached to the surface of the glove. This is especially the case when continuous and frequent wearing of gloves is required. In addition, hand washing is necessary after the use of powdered gloves. Frequent hand washing to remove powder may also cause excess dryness of the skin.

The need for disposable gloves that can prevent adverse side effects caused by extensive use is apparent. Various patents disclose different types of gloves that contain moisturizers such as lotions that contact human skin during glove use. For example, U.S. Pat. No. 5,614,202 discloses a moisturizing glove that contains a middle layer saturated with lotion. The porous inner layer allows the lotion to pass through and contact the skin. U.S. Pat. Nos. 4,186,445 and 4,185,330 disclose gloves that have an inner lining made of a lotion absorbent material. By impregnating the lotion onto the absorbent material, the lotion can condition the hands during application of the gloves.

A common feature of the above disclosures is the use of multiple layers in the glove design. Compared to single layer disposable gloves, the complex design of multiple layer gloves makes production far more costly. More importantly, the thickness of the layers and the complicated structures of the gloves hinder hand flexibility when the glove wearer tries to pick up and manipulate objects. Such multiple layer designs are suitable for moisturizing hands, but are not suitable for manipulating objects, especially for professions that require handling of fine tasks with precision.

Disposable gloves are generally made from one of three types of materials: natural rubber latex, acrylonitrile, or polyvinyl chloride.

Natural rubber latex is sensitive to oil-based substances. Prolonged contact between latex and an oil-based substance can adversely affect the durability and flexibility of the latex material. Most commercially available lotions contain oil-based substances. The use of conventional lotions can thus substantially shorten the shelf life of a natural rubber glove.

Several patents also disclose a glove containing aloe vera as a moisturizer. For example, U.S. Pat. Nos. 6,274,154, 6423,328, and 6,630,152 disclose an aloe vera glove and manufacturing method thereof. However, aloe vera does not provide the anti-inflammatory and astringent properties desired by some users.

There is therefore a need for low cost disposable gloves that can apply moisturizing and other therapeutic properties to the hands during glove use while at the same time retaining the characteristics and functions of conventional single layer gloves.

### SUMMARY

In accordance with one embodiment of the present invention, a glove is disclosed, comprising a layer having an exterior surface and an interior surface opposite from the exterior surface, the interior surface forming a cavity for receiving a hand. The glove further includes a coating on the interior surface of the layer, the coating including a dehydrated cucumber extract.

In accordance with another embodiment of the present invention, a glove similar to that disclosed above includes a coating on the interior surface of the layer, the coating including a cucumber extract gel.

In accordance with yet another embodiment of the present invention, a glove similar to that disclosed above includes a coating on the interior surface of the layer, the coating including glycerin.

In accordance with yet another embodiment of the present invention, a method of manufacturing a glove is disclosed, comprising: forming a glove on a mold; applying a solution that includes cucumber extract onto a portion of the glove; and at least partially drying the solution that has been applied onto the glove in order to form a cucumber layer on the glove.

In accordance with yet another embodiment of the present invention, a method similar to that disclosed above includes applying a solution that includes glycerin onto a portion of the glove and at least partially drying the solution in order to form a glycerin layer on the glove.

These and other features and advantages of the present invention will be more readily apparent from the detailed description of the embodiments set forth below taken in conjunction with the accompanying drawings.

### BRIEF DESCRIPTION OF DRAWINGS

FIG. 1A is a perspective view of a therapeutic glove in accordance with one embodiment of the present invention.

FIG. 1B is a sectional view of FIG. 1A taken along the line I-I.

FIG. 2 is a flow diagram that illustrates a method for manufacturing therapeutic gloves according to an embodiment of the present invention.

FIG. 3 is a flow diagram that illustrates a method for manufacturing therapeutic gloves that is integrated with, and includes, the manufacture of the underlying gloves themselves in accordance with another embodiment of the present invention.

Use of the same reference symbols in different figures indicates similar or identical items. It is further noted that the drawings may not be drawn to scale.

### DETAILED DESCRIPTION

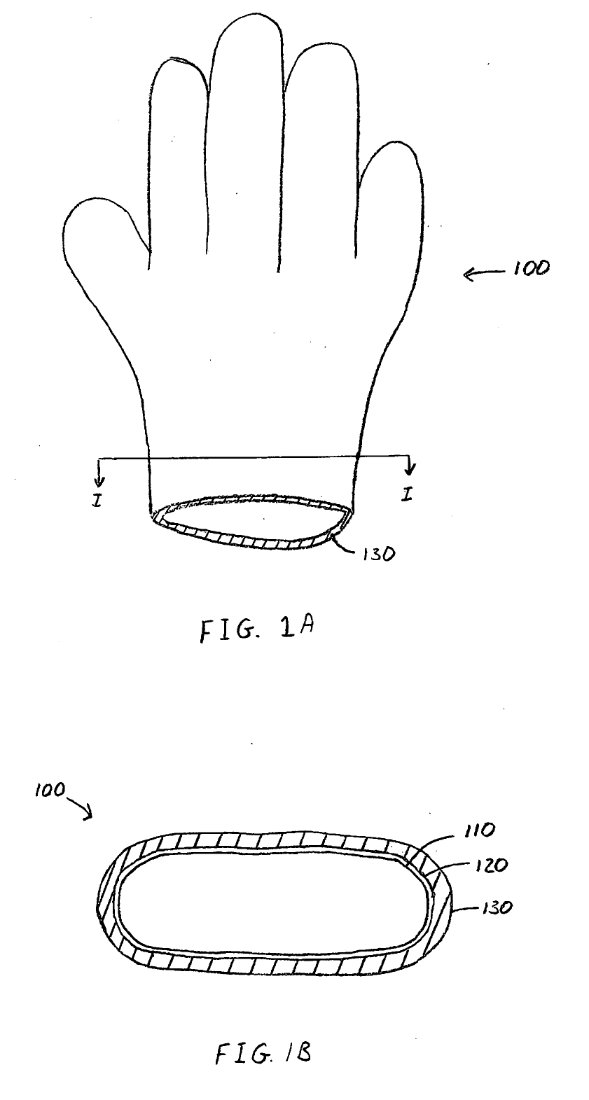

An embodiment of the present invention is illustrated in FIGS. 1A and 1B as a glove 100 formed by a glove layer 130. Glove layer 130 has an exterior surface and an interior surface with the interior surface forming a cavity for receiving a hand. FIG. 1B is a cross-sectional view of the glove in FIG. 1A taken along the line I-I, and illustrates a therapeutic layer 110 evenly coated on an interior surface 120 of glove layer 130.

In one example, therapeutic layer 110 includes a cucumber extract (cucumis sativus extract) in a dehydrated state. In another example, therapeutic layer 110 is a coat of cucumber extract gel consisting of cucumber extract and water. In a further example, therapeutic layer 110 includes cucumber extract supplemented with vitamin E, glycerin, lanolin, natural moisturizing factor (NMF), ginseng extract, and/or algae extract.

In yet further examples, therapeutic layer 110 consists of or comprises glycerin (also known as glycerol), which is a humectant that will attract moisture to the skin making it soft and supple. Glycerin is also highly “hygroscopic” which means that it absorbs water from the air. The glycerin may be supplemented with cucumber extract, vitamin E, lanolin, NMF, ginseng extract, and/or algae extract.

In one embodiment, the glove advantageously retains the features of a disposable examination glove, which is simple and convenient to use and allows the wearer to handle fine tasks with precision. Another embodiment of the present invention is a manufacturing method for modifying a disposable glove by applying a cucumber and/or glycerin coating on the inner surface of the glove. The glove is coated with therapeutic layer 110, in one example through dehydration that is accomplished by a well-controlled heating process.

Glove layer 130 may be made of various materials, in one example being formed of a fluid-impermeable material. Resinous materials such as vinyl, or polymer materials such as acrylonitrile, are common choices. Three commonly used materials for making disposable gloves are natural rubber latex, acrylonitrile, and polyvinyl chloride.

In one embodiment, glove layer 130 is made of natural rubber latex. Since natural rubber latex is sensitive to oil-based substances, gloves made of natural rubber latex should not be exposed to oil-based substances. In this embodiment, cucumber extract or glycerin is used to coat the gloves and the extract does not contain any detectable oil-based substances. Advantageously, coating gloves with cucumber extract or glycerin does not affect the glove's shelf life. In another embodiment glove layer 130 is made of acrylonitrile polymer.

Furthermore, cucumber is soluble in water and is a powerful anti-inflammatory, skin soothing, and skin conditioning agent. Cucumber also displays highly effective moisturizing and astringent properties, more so than other materials such as aloe vera.

Glove 100 retains the characteristics of a disposable glove without any visible modification and is easy and convenient to use. The affiliation between therapeutic layer 110 and the inner surface 120 of base glove layer 130 is through a force provided by dehydration in one example and adhesive properties of the therapeutic layer 110 in another example. Such affiliation is loosened when sweat comes in contact with therapeutic layer 110. The longer a glove is worn, the more likely the hand will sweat, and consequently more of the therapeutic layer, such as cucumber or glycerin, will be dissolved and disassociated from the glove surface and applied to the hand. Thus, the active ingredients in cucumber or glycerin can effectively condition the skin in contact with the therapeutic layer.

In one embodiment, 100% cucumber extract coats the inner surface of the glove. Cucumber extract is evenly and uniformly distributed on the inner surface of the glove at a thickness of about 0.01 millimeter in one example. The association between cucumber extract and the base glove layer surface is achieved by a noncovalent force provided through dehydration in one example.

The method of manufacturing therapeutic gloves includes treating a commercially available disposable glove to eliminate residue powders, soluble substances, and microorganisms, turning the glove inside out, and providing a therapeutic layer on the interior surface of the glove. In one example, the cucumber extract may be provided by spraying with or dipping in a cucumber solution, and heating the glove to cause water to evaporate.

A glove is preferably first treated with a chlorine solution or chlorine gas. Chlorine solution can help to sterilize the glove, to wash off powders, and most importantly for natural latex gloves, to dissolve residual proteins that could potentially trigger severe allergic reactions among repeat users. After the outside surface of the glove is treated with the chlorine solution, it is turned inside out, and the glove is again treated with the chlorine solution. The residue chlorine is neutralized by using ammonia and the glove is then dried.

A therapeutic solution, in one example a cucumber or glycerin solution, will then be prepared. In one example, 100% concentrated cucumber gel is dissolved in distilled water to generate a cucumber solution. In a further example, the concentration of the solution may be about 20% by weight. In yet a further example, the cucumber solution may include cucumber extract and supplements vitamin E, glycerin, lanolin, natural moisturizing factor (NMF), ginseng extract, and/or algae extract. To associate cucumber extract with the surface of the glove, cucumber solution can be sprayed onto the surface of the glove. Alternatively, the glove can be immersed into the cucumber solution by dipping. A glycerin solution may be similarly prepared and applied.

In one embodiment, the dipping process is accomplished by grouping a number of gloves in a batch to achieve higher manufacturing efficiency. In one example, the gloves are immersed in the solution for at least 10 minutes to allow adequate absorbency.

Cucumber extract is attached to the surface of the glove through a controlled dehydration process in one example. The water in the cucumber solution is caused to evaporate through heating. Although higher temperatures will cause water to evaporate more quickly, excess heat may damage the base glove layer. For example, gloves exposed to excessive heat of over 70° C. may turn brownish and become brittle. To shorten the heat exposure time, a heating oven may be preheated to about 45° C. before the gloves are introduced. The oven has a temperature control mechanism to maintain a maximum temperature. In one example, the maximum temperature is set at approximately 65° C. and the heating process lasts from about 35 to 40 minutes. The dehydration process provides an affiliation force so that cucumber extract can remain associated with the glove surface for an extensive period of time.

Even distribution of the therapeutic solution (e.g., cucumber extract or glycerin) on the glove surface maximizes therapeutic treatment of the hand and minimizes contact between the skin and the glove's composite material. Stationary drying is not preferred because the therapeutic solution tends to flow in the direction of the force of gravity. In one embodiment, the heating oven has a device to tumble dry the glove during the heating process to make cucumber or glycerin solution distribute evenly on the glove surface and to form a uniform coating of cucumber extract or glycerin after heating.

Afterward the gloves are cooled to room temperature. The gloves are then inverted so that the surface with the therapeutic layer faces inward.

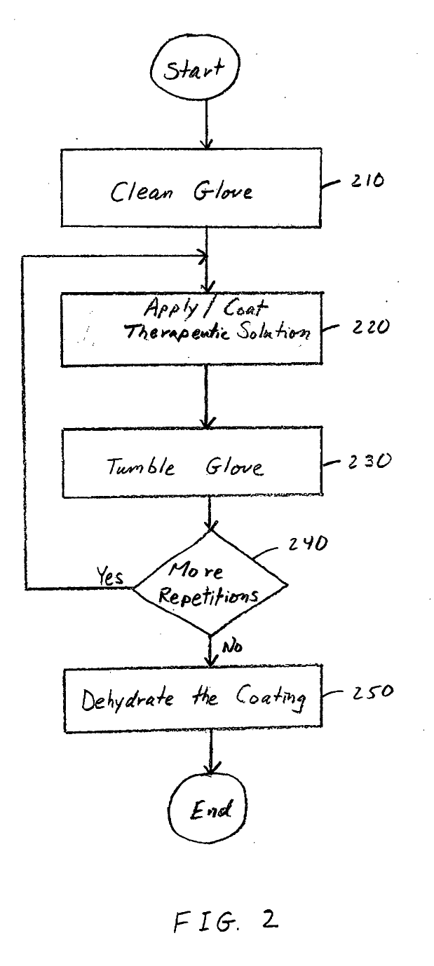

FIG. 2 is a flow diagram that illustrates a method for manufacturing therapeutic gloves according to an embodiment of the present invention. The application of therapeutic solution to gloves begins with gloves that are clean and free of protein residue, powder, or other surface contaminants. Therefore, the method preferably begins with a step 210 of cleaning the gloves to remove such contaminants. Next, therapeutic solution is applied to the gloves (step 220), in one example by spraying a batch of clean loose gloves that are arranged inside out. The gloves are tumbled (step 230) for greater distribution of therapeutic solution and even coating of the therapeutic layer. In one example, the tumbling of the gloves in the step 230 occurs, or continues to occur, after the spraying of the therapeutic solution in the step 220 has already stopped. The steps 220 and 230 are then preferably repeated for a desired number of iterations (as shown by decision box 240 in FIG. 2). After the last iteration of the steps 220 and 230 of applying the therapeutic solution, the gloves are dried (step 250).

The optional (but preferred) step 210 of cleaning the gloves of surface contaminants can be performed using any competent technique (including any conventional technique). For example, as discussed above, a chlorine solution may be used, and the chlorine solution itself is preferably neutralized and cleaned away at the end of the cleaning step. Cleaning items such as gloves of surface contaminants is known, and the specifics of such cleaning will be readily apparent depending on the particular type of cleaning equipment being used. In one example, for a sufficiently large commercial chlorine washer, a batch of about 3000 to 4000 gloves may be washed using any conventional cycle, for example a cycle of about 20 to 30 minutes. Optionally, for extra assurance of cleanliness, the batch of gloves may be further rinsed with water, preferably in a separate commercial washing tank, for example first with hot water and then with cold (e.g., room-temperature) water for any desired amount of time, for example about 20 to 30 minutes or more. The water is preferably drained well from the gloves prior to application of the therapeutic solution. For example, the gloves may be spun dry in the commercial washing tank in a conventional manner.

The steps 220, 230, and 250 are all performed within a commercial heat tumble dryer, for example, as follows. After the optional water bath at the end of the optional cleaning step 210, the gloves are removed from the water bath and dumped into the heat tumble dryer. The dryer then starts tumbling the gloves. Preferably, the tumbling is accompanied by heating of the gloves by hot air and continues until the gloves are dry or mostly dry. Then, a spray nozzle configured to spray therapeutic solution as a fine mist starts spraying the therapeutic solution onto the gloves in the dryer. During the spraying, the tumbling may either continue or may continue at a slower pace or may be stopped, and heating of the air may be continued or reduced or stopped. Depending on the level of integration between the spray nozzle and the dryer, the door of the dryer may be opened to allow access to the spray nozzle during spraying. After a period of spraying, the spraying stops and the tumbling continues, or resumes, preferably accompanied by resumed, or continued, heating of the air. The spraying and tumbling are repeated for several iterations. After the last iteration of spraying, the gloves are dried, preferably by tumbling with heating until the gloves are dry. The number and duration of iterations and the amount of solution used should be chosen to be sufficient to leave at least a desired minimum thickness, and/or no more than a desired maximum thickness, of the therapeutic layer on substantially every glove given the particular dryer and spray nozzle configuration.

Preferably, the method as illustrated in FIG. 2 is performed and completed using only two or only three holding containers in which washing, spraying, or tumbling are actually performed. If two containers are used, they would be the chlorine washer and the heat tumble dryer. If three containers are used, they would be the chlorine washer, the water washer, and the heat tumble dryer.

FIG. 2 can also serve as a flow diagram for the earlier-discussed embodiment of the present invention that is a method that uses immersion (e.g., dipping) to apply therapeutic solution. If FIG. 2 is interpreted to describe the method that uses immersion, then preferably the decision box 240 reflects having only a single iteration of immersing (the step 220), and the box for step 230 can be interpreted to refer to agitation of the immersion tank, for example, in the manner of a washing machine. After the immersion (e.g., step 220), the step 250 refers to tumble drying, as has been discussed earlier. If FIG. 2 is used to describe the immersion method, then preferably an extra holding container would be used, namely, an immersion tank that contains the therapeutic solution. Thus, if the method is embodied so as to use immersion, then the method is preferably performed and completed using only three or only four holding containers in which washing, immersion, or tumbling are actually performed. If three containers are used, they would be the chlorine washer, the immersion tank for therapeutic solution, and the heat tumble dryer. If four containers are used, they would be the chlorine washer, the washer for water, the immersion tank for therapeutic solution, and the heat tumble dryer.

In another embodiment of the present invention, a method for manufacturing therapeutic gloves is integrated with, and/or includes, the manufacturing of the underlying gloves themselves. This other embodiment is especially preferred for producing large quantities of therapeutic gloves efficiently.

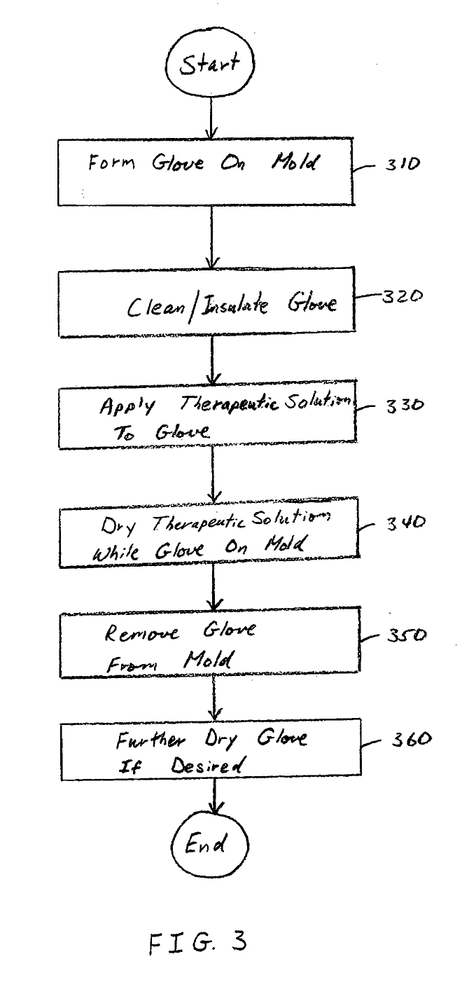

FIG. 3 is a flow diagram that illustrates another method for manufacturing therapeutic gloves that is integrated with, and includes, the manufacturing of the underlying gloves themselves. Preferably, the method is fully automated within a production line. In a step 310, gloves are formed on molds using any conventional technique. The forming and formed gloves undergo processing on the molds in the step 320 using, for example, conventional processing. In a step 330, a therapeutic solution, for example, the cucumber or glycerin solution discussed earlier, is applied to the gloves while the gloves are still on the mold. The application of the solution can be via any competent technique, for example, spraying, immersing, pouring, overfilling, dipping, and the like, which are not mutually exclusive techniques. In a step 340, the therapeutic solution that coats the gloves undergoes at least partial, and preferably full or at least substantial, dehydration. Next, in a step 350, the gloves are removed from the molds. Optionally, after removal from the molds, the gloves are further dried and cured by heat in a step 360.

In the step 310, the gloves formed on the mold are preferably considered to be inside out such that the interior of each glove, as later to be worn on the hand, faces outward. The gloves are formed and processed using whatever technique is competent to produce a glove of the desired material. The preferred material is natural rubber latex.

In the step 320, after a glove is formed and while on the mold, the later hand-facing surface of the glove is preferably made safer, and/or easier to slide during donning, for later contact with hands, either by cleaning off any residual proteins, chemicals, and the like, for example using chlorine, or by coating the surface with a thin insulating layer that will attempt to insulate the hand from contact with the residual proteins, chemicals, and the like during wearing of the glove. By being cleaned, the glove is likely to be slicker and easier to slide over skin during donning, especially if the glove is made of natural rubber latex. Similarly, the insulating layer is preferably made of a substance that is more slippery than the underlying glove. For example, even if the glove is a vinyl glove of a type that is not made significantly safer or more slippery by cleaning, it may still be coated with an insulating layer to decrease friction and thereby be made easier to don. The insulating layer is, for example, a polymer layer of silicone or polyurethane.

In the step 330, therapeutic solution, such as has already been described, is applied to the gloves while the gloves are still on the molds, either by dipping or by spraying. If spraying is used, it should be thorough enough so as to leave a desired amount of solution on the gloves' interior surface, for example, an amount comparable to that which would be obtained from dipping.

In the step 340, the gloves undergo at least partial, and preferably full or at least substantial, dehydration. For example, fanned heated air may be blown across the gloves on the molds. Especially for natural rubber latex gloves, the air is preferably not more than about 80° C., and even more preferably, the air is not more than about 65° C. The therapeutic coating is sufficiently dried to provide sufficient adhesion between the therapeutic layer and the glove surface so that the coated glove can withstand subsequent step 350, in which the gloves are stripped from the molds by conventional techniques.

In the optional step 360, the loose gloves are further cured, and their therapeutic coatings are even further dehydrated by heat, for example in a dryer as has been discussed earlier.

The forming and processing of gloves on molds in the step 310, in one example, includes on an automatic production line: cleaning porcelain formers (molds) using hot water (for example, about 40° C. to about 100° C.); drying the porcelain formers in hot air (for example, at about 40° C. to about 100° C.); dipping the formers in coagulant (for example, at about 40° C. to about 70° C.); drying the coagulant on the formers in hot air (for example, at about 35° C. to about 140° C.); dipping the coagulant-coated formers in latex (for example, at about 25° C. to about 45° C.); curing the latex on the formers in hot air (for example, at about 60° C. to about 140° C.); leaching the gloves on the formers; beading the edge of the gloves on the formers; and then making the glove surfaces safer, and easier to don, for later contact with hands, either by cleaning or by coating the surface, as discussed above.

If a cleaning step is used the method also includes: further curing (for example, at about 80° C. to about 140° C.); rinsing with cold water (for example, at no more than room temperature); chlorination (for example, at no more than about 30° C.); preferably preceded by further rinsing with cold water (for example, at no more than room temperature); neutralization; further rinsing (for example, with hot followed by cold water); and dehydration and further curing in hot air. Alternatively, if an insulation coating step is used, the method also includes: drying in hot air (for example, at about 80° C. to about 150° C.); coating with polymer (for example, at no more than about 45° C.); and further drying and curing in hot air (for example, at about 80° C. to about 150° C.)

The above-described embodiments of the present invention are merely meant to be illustrative and not limiting. Various changes and modifications may be made without departing from this invention in its broader aspects. For example, in addition to the preferred natural rubber latex, the present invention may be embodied as therapeutic gloves of acrylonitrile, polyvinyl chloride, polyurethane, chloroprene, neoprene, butadiene, or the like. Accordingly, the invention is not limited to particular structures, dimensions, or process parameters. Therefore, the appended claims encompass all such changes and modifications as falling within the true spirit and scope of this invention.

We claim:

1. A glove, comprising:
a layer having an exterior surface and an interior surface opposite from the exterior surface, the interior surface forming a cavity for receiving a hand; and
a coating on the interior surface of the layer, the coating including a dehydrated cucumber extract.

2. The glove of claim 1, wherein the layer is fluid-impermeable.

3. The glove of claim 1, wherein the layer is made from a material selected from the group consisting of a natural rubber latex and an acrylonitrile polymer.

4. The glove of claim 1, wherein the coating is substantially free of oil-based substances.

5. The glove of claim 1, wherein the coating has a thickness of about 0.1 mm.

6. The glove of claim 1, wherein the coating consists of cucumber extract.

7. The glove of claim 1, wherein the coating is evenly distributed on the interior surface of the layer.

8. The glove of claim 1, wherein the coating is in direct contact with the hand during donning or removing of the glove.

9. The glove of claim 1, wherein the coating consists of cucumber extract and supplements selected from the group consisting of Vitamin E, glycerin, lanolin, natural moisturizing factor (NMF), ginseng extract, and algae extract.

10. A glove, comprising:
a layer having an exterior surface and an interior surface opposite from the exterior surface, the interior surface forming a cavity for receiving a hand; and
a coating on the interior surface of the layer, the coating including a cucumber extract gel.

11. The glove of claim 10, wherein the layer is fluid-impermeable.

12. The glove of claim 10, wherein the layer is made from a material selected from the group consisting of a natural rubber latex and an acrylonitrile polymer.

13. The glove of claim 10, wherein the coating is substantially free of oil-based substances.

14. The glove of claim 10, wherein the coating is evenly distributed on the interior surface of the layer.

15. The glove of claim 10, wherein the coating is in direct contact with the hand during donning or removing of the glove.

16. The glove of claim 10, wherein the coating consists of cucumber extract gel.

17. The glove of claim 10, wherein the cucumber extract gel consists of cucumber extract and water.

18. The glove of claim 10, wherein the coating consists of cucumber extract gel and supplements selected from the group consisting of Vitamin E, glycerin, lanolin, natural moisturizing factor (NMF), ginseng extract, and algae extract.

19. A glove, comprising:
a layer having an exterior surface and an interior surface opposite from the exterior surface, the interior surface forming a cavity for receiving a hand; and
a coating on the interior surface of the layer, the coating including glycerin.

20. The glove of claim 19, wherein the layer is fluid-impermeable.

21. The glove of claim 19, wherein the layer is made from a material selected from the group consisting of a natural rubber latex and an acrylonitrile polymer.

22. The glove of claim 19, wherein the coating is substantially free of oil-based substances.

23. The glove of claim 19, wherein the coating is evenly distributed on the interior surface of the layer.

24. The glove of claim 19, wherein the coating is in direct contact with the hand during donning or removing of the glove.

25. The glove of claim 19, wherein the coating consists of glycerin.

26. The glove of claim 19, wherein the coating consists of glycerin and water.

27. The glove of claim 19, wherein the coating consists of glycerin and supplements selected from the group consisting of Vitamin E, lanolin, natural moisturizing factor (NMF), cucumber extract, ginseng extract, and algae extract.

28. A method of manufacturing a glove, comprising:
forming a glove on a mold;
applying a solution that includes cucumber extract onto a portion of the glove; and
at least partially drying the solution that has been applied onto the glove in order to form a cucumber layer on the glove.

29. The method of claim 28, wherein applying the solution is accomplished while the glove is on the mold.

30. The method of claim 28, wherein applying the solution is accomplished while the glove is no longer on the mold.

31. The method of claim 28, wherein applying the solution includes spraying the solution onto the glove.

32. The method of claim 28, wherein applying the solution includes dipping the glove into a quantity of the solution.

33. The method of claim 28, wherein at least partially drying the solution includes blowing heated air onto the glove.

34. The method of claim 28, wherein the solution consists of cucumber extract and water.

35. The method of claim 28, wherein the solution consists of cucumber extract, water, and supplements selected from the group consisting of Vitamin E, glycerin, lanolin, natural moisturizing factor (NMF), ginseng extract, and algae extract.

36. The method of claim 28, wherein at least partially drying the solution that has been applied onto the glove is accomplished while the glove is on the mold.

37. The method of claim 28, wherein at least partially drying the solution that has been applied onto the glove is accomplished after the glove is no longer on the mold.

38. The method of claim 28, wherein drying the solution includes tumbling the glove in a heat dryer.

39. A method of manufacturing a glove, comprising:
forming a glove on a mold;
applying a solution that includes glycerin onto a portion of the glove; and
at least partially drying the solution that has been applied onto the glove in order to form a glycerin layer on the glove.

40. The method of claim 39, wherein applying the solution is accomplished while the glove is on the mold.

41. The method of claim 39, wherein applying the solution is accomplished while the glove is no longer on the mold.

42. The method of claim 39, wherein applying the solution includes spraying the solution onto the glove.

43. The method of claim 39, wherein applying the solution includes dipping the glove into a quantity of the solution.

44. The method of claim 39, wherein at least partially drying the solution includes blowing heated air onto the glove.

45. The method of claim 39, wherein the solution consists of glycerin and water.

46. The method of claim 39, wherein the solution consists of glycerin, water, and supplements selected from the group consisting of Vitamin E, lanolin, natural moisturizing factor (NMF), cucumber extract, ginseng extract, and algae extract.

47. The method of claim 39, wherein at least partially drying the solution that has been applied onto the glove is accomplished while the glove is on the mold.

48. The method of claim 39, wherein at least partially drying the solution that has been applied onto the glove is accomplished after the glove is no longer on the mold.

49. The method of claim 39, wherein drying the solution includes tumbling the glove in a heat dryer.
